SEQUENCE LISTING (22 sequences)

SEQ ID NO: 1 - protein, 61 aa, Homo sapiens
IQKAIRGIGTDEKMLISILTERSNAQRQLIVKEYQAAYGKELKDDLKGDLSGHFEHLMVAL

SEQ ID NO: 2 - protein, 61 aa, Homo sapiens
LHRALKGIGTDEFTLNRIMVSRSEIDLLDIRTEFKKHYGYSLYSAIKSDTSGDYEITLLKI

SEQ ID NO: 3 - protein, 61 aa, Homo sapiens
LKKSMKGAGTNEDALIEILTTRTSRQMKDISQAYYTVYKKSLGDDISSETSGDFRKALLTL

SEQ ID NO: 4 - protein, 61 aa, Homo sapiens
YKAGENRWGTDEDKFTEILCLRSFPQLKLTFDEYRNISQKDIVDSIKGELSGHFEDLLLAI

SEQ ID NO: 5 - protein, 323 aa, Homo sapiens
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 6 - protein, 81 aa, Artificial
  Synthetic Construct - Recombinant D1
MGHHHHHHHHSPSVDAEAIQKAIRGIGTDEKMLISILTERSNAQRQLIVKEYQAAYGKELKDDLKGDLSGHFEHLMVALVT

SEQ ID NO: 7 - protein, 79 aa, Artificial
  Synthetic Construct - Recombinant D2
MGHHHHHHHHAVFDAKQLKKSMKGAGTNEDALIEILTTRTSRQMKDISQAYYTVYKKSLGDDISSETSGDFRKALLTLA

SEQ ID NO: 8 - protein, 85 aa, Artificial
  Synthetic Construct - Recombinant D3
MGHHHHHHHHDEHLAKQDAQILYKAGENRWGTDEDKFTEILCLRSFPQLKLTFDEYRNISQKDIVDSIKGELSGHFEDLLLAIVN

SEQ ID NO: 9 - protein, 82 aa, Artificial
  Synthetic Construct - Recombinant D4
MGHHHHHHHHAFLAERLHRALKGIGTDEFTLNRIMVSRSEIDLLDIRTEFKKHYGYSLYSAIKSDTSGDYEITLLKICGGDD

SEQ ID NO: 10 - protein, 17 aa, Homo sapiens
SNAQRQLIVKEYQAAYG

SEQ ID NO: 11 - protein, 11 aa, Homo sapiens
LIVKEYQAAYG

SEQ ID NO: 12 - protein, 12 aa, Homo sapiens
IVKEYQAAYGKE

SEQ ID NO: 13 - protein, 8 aa, Homo sapiens
KEYQAAYG

SEQ ID NO: 14 - protein, 9 aa, Homo sapiens
DLSGHFEHL

SEQ ID NO: 15 - protein, 7 aa, Homo sapiens
LSGHFEH

SEQ ID NO: 16 - protein, 20 aa, Homo sapiens
ASIWVGHRGTVRDYPDFSPS

SEQ ID NO: 17 - protein, 18 aa, Homo sapiens
SIWVGHRGTVRDYPDFSP

SEQ ID NO: 18 - protein, 4 aa, Homo sapiens
YPDF

SEQ ID NO: 19 - protein, 38 aa, Homo sapiens
KEYQAAYGKELKDDLKGDLSGHFEHLMVALVTPPAVFD

SEQ ID NO: 20 - protein, 56 aa, Homo sapiens
QKAIRGIGTDEKMLISILTERSNAQRQLIVKEYQAAYGKELKDDLKGDLSGHFEHL

SEQ ID NO: 21 - protein, 26 aa, Homo sapiens
KEYQAAYGKELKDDLKGDLSGHFEHL

SEQ ID NO: 22 - protein, 17 aa, Homo sapiens
KEYQAAYGKELKDDLKG

(19) United States
(12) Patent Application Publication
(10) Pub. No.: US 2014/0017710 A1
(43) Pub. Date: Jan. 16, 2014

(54) PROCESS AND KIT FOR IN VITRO DIAGNOSIS OF A PROSTATE CANCER

(71) Applicants: Yasemin Ataman-Onal, Reyrieux (FR); Sandrine Busseret, Lyon (FR); Genevieve Choquet-Kastylevsky, Francheville (FR); Emanuelle Gallet-Gorius, Echalas (FR); Gaspard Gervasi, Lyon (FR); Michele Guillotte, Lozanne (FR); Celine Hamelin, Rontalon (FR)

(72) Inventors: Yasemin Ataman-Onal, Reyrieux (FR); Sandrine Busseret, Lyon (FR); Genevieve Choquet-Kastylevsky, Francheville (FR); Emanuelle Gallet-Gorius, Echalas (FR); Gaspard Gervasi, Lyon (FR); Michele Guillotte, Lozanne (FR); Celine Hamelin, Rontalon (FR)

(73) Assignee: BIOMERIEUX, Marcy L'Etoile (FR)

(21) Appl. No.: 13/990,211

(22) PCT Filed: Dec. 7, 2011
(86) PCT No.: PCT/FR11/52893

§ 371 Date: May 29, 2013

(30) Foreign Application Priority Data
Dec. 8, 2010 (FR) 1060221

(51) Int. Cl. G01N 33/68

(52) U.S. Cl. CPC G01N 33/6893
USPC 435/7.92

(57) ABSTRACT

The object of the present invention is a process for in vitro diagnosis of a prostate cancer, according to which a urine sample to be analysed is contacted with two antibodies, a capture antibody and a detection antibody, one of the two antibodies being directed against the first repeat domain of native human Annexin A3 identified as SEQ ID NO: 1, and the other of the two antibodies being directed against the fourth repeat domain of native human Annexin A3, identified as SEQ ID NO: 2, as well as a kit for implementing the process.

The object of the present invention is a process and a kit for in vitro diagnosis of prostate cancer.

Prostate cancer is the most frequent type of cancer in men. Prostate cancer has the particularity of a very slow development. Nonetheless, in spite of its moderate biological aggressiveness compared to other types of cancers, it is the second most fatal cancer in men, after lung cancer, and on a par with colorectal cancer (1). The severity of the cancer depends on the extent of the tumour (local, with neighbouring or remote metastases) and the type of cancer cells, i.e. their degree of malignity. There are curative solutions, especially for early stages of cancer which are locally limited, such as radiotherapy and/or surgery. So it is crucial to ensure availability of a test enabling a prostate cancer diagnosis to be performed as early as possible and reliably.

Prostate cancer screening is possible by means of observing increased concentration of a protein in the blood, prostate specific antigen or PSA. PSA is produced by the prostate, and is therefore a specific marker of the organ, but it is not specific to the tumour. That is why a high result in a PSA quantification test does not necessarily mean that there is a cancer. Indeed, a quantity of more than four nanograms per millilitre of this protein in the blood is associated with a prostate cancer in 25% of cases, and with another prostate disorder in 75% of cases, especially with benign hypertrophy (BPH), an inflammation or an infection of the prostate. Furthermore, although it is relatively sensitive for initial diagnoses and for patient monitoring, this test cannot detect all cases of prostate cancer.

More recently, another marker was described as being significantly associated with the progression and the various stages of prostate diseases and prostate cancer. This is Annexin 3 (ANXA3), which belongs to the Annexin family. The Annexin family proteins have the shared characteristic of being able to bind to phospholipid membranes in the presence of calcium. The members of this protein family share structural similarities enabling them to do so. Hence every Annexin contains in its protein sequence so-called “annexin repeat” domains, which are 4 or 8 in number (4 for Annexin A3), and which are approximately 70 amino acids in length (2). Every repeat domain, also known as the endonexin domain, is folded into 5 alpha helices and contains a type 2 characteristic motif (GxGT-[38 residues]-D/E), which enables binding of Ca2+ ions. Annexins in the animal kingdom have variable N-terminus domains, which often bear the functional specificities of the proteins. Crystallographic analyses show that the secondary and tertiary structures of annexins are maintained, even if the identicalness of the amino acid sequences does not exceed 45 to 55%. The 4 repeat domains form a structure resembling a disc, with a slightly convex surface on which the Ca²⁺ binding sites are located, and a concave surface where the N- and C-termini of the protein are in proximity.

Annexin A3 is a rare annexin, which has only a low expression, or no expression at all, in most cell types. Conversely, it is highly abundant in human neutrophils, where it represents approximately 1% of cytosolic proteins (3, 4). Initially, two Annexin A3 isoforms were described; one with apparent molecular mass 33 kDa, present mainly in neutrophils, and the other with apparent molecular mass 36 kDa, found in monocytes. The difference in molecular mass between the two isoforms is not a result of a post-translational modification such as phosphorylation or glycosylation. Very recently, in 2010, it was demonstrated that an alternative splicing phenomenon could be a mechanism explaining the presence of these two isoforms. Thus, the authors identified two variants of DNA complementary to Annexin A3: the first, 973 base pairs (bp) in length, encodes for a whole protein of 323 amino acids, corresponding to the 36 kDa isoform; the second, 885 bp in length, does not contain exon III, due to alternative splicing. If this exon is not present, the open reading frame is interrupted, and translation becomes possible only from the ATG codon present in exon IV. Consequently, the first 39 amino acids in the protein are not expressed, resulting in a protein truncated at the N-terminus by 284 amino acids, corresponding to the 33 kDa isoform (5).

Furthermore, Western blot analysis after 2-dimensional electrophoresis (2DE-WB) shows that the 36 kDa isoform migrates in the form of 4 to 6 spots, with different isoelectric points. The 36 kDa isoform identified by a 1-dimensional Western blot actually corresponds to a group of heterogeneous isoforms (5). It is the 36 kDa Annexin A3 whose expression level falls in case of prostate cancer (5-7).

Although Annexin A3 is recognised as a marker for prostate cancer, the processes for detecting it which have been described until now either lack specificity, because the antibodies used have cross reactions with other annexins (WO 2006/125580), or lack sensitivity for its detection in a complex medium such as urine (WO 2007/141043). In general terms, prior processes have lacked robustness.

Surprisingly, the inventors found that to compensate for the above-mentioned shortcomings, it was necessary to use a pair of antibodies, one of which is directed against the first repeat domain of native human Annexin A3, the sequence of which is identified as SEQ ID NO: 1 (Annexin A3 amino acids 27-87, according to the numbering in the UniProtKB database for complete human ANXA3, accession No. P12429), and the other is directed against the fourth repeat domain of native human Annexin A3, the sequence of which is identified as SEQ ID NO: 2 (amino acids 258-318, according to the numbering in the UniProtKB database for complete human ANXA3, accession No. P12429).

In the description below, the sequence of the first repeat domain of native human Annexin A3 is identified as SEQ ID NO: 1, and the sequence of the fourth repeat domain of native human Annexin A3 is identified as SEQ ID NO: 2, the sequence of the second repeat domain of native human Annexin A3 is identified as SEQ ID NO: 3 and the sequence of the third repeat domain of native human Annexin A3 is identified as SEQ ID NO: 4. The sequence identified as SEQ ID NO: 5 corresponds to the amino acid sequence of complete native human Annexin A3 (UniProtKB accession No. P12429).

The object of the present invention is also a process for in vitro diagnosis of a prostate cancer, according to which a urine sample to be analysed is contacted with two antibodies; a capture antibody and a detection antibody, one of the two antibodies being directed against the first repeat domain of native human Annexin A3, the sequence of which is identified as SEQ ID NO: 1, and the other of the two antibodies being directed against the fourth repeat domain of native human Annexin A3, the sequence of which is identified as SEQ ID NO: 2.

In particular, the antibody directed against the first repeat domain of native human Annexin A3 is chosen from the antibodies directed against an epitope, the amino acid sequence of which comprises at least 7 consecutive amino acids, preferably at least 8 or at least 9 amino acids, or even at least 12 consecutive amino acids, and no more than 17 consecutive amino acids of SEQ ID NO: 1, among which can be mentioned, by way of preference, the antibodies which are directed against a polypeptide included in SEQ ID NO: 1, the amino acid sequence of which is selected from the sequences below:

### SNAQRQLIVKEYQAAYG (SEQ ID NO: 10),

### LIVKEYQAAYG (SEQ ID NO: 11)

### IVKEYQAAYGKE (SEQ ID NO: 12),

### KEYQAAYG (SEQ ID NO: 13),

### DLSGHFEHL (SEQ ID NO: 14),

### LSGHFEH (SEQ ID NO: 15),

and
KEYQAAYGKELKDDLKG (SEQ ID NO: 22), provided that the amino acid sequence SEQ ID NO: 22 is fused on the N-terminus side to a sequence of at least 30 amino acids.

Among the antibodies directed against the fourth repeat domain of native human Annexin A3, it is possible to mention the antibodies directed against an epitope, the amino acid sequence of which comprises at least 7 consecutive amino acids and no more than 50, preferably no more than 45 consecutive amino acids, of SEQ ID NO: 2.

In particular the antibodies directed against the fourth repeat domain of native human Annexin A3 are chosen from antibodies directed against an epitope which:
is included in an amino acid sequence corresponding to the amino acid sequence starting at residue 3, and ending at residue 49 of SEQ ID NO: 2,
comprises in position 6 of SEQ ID NO: 2 a Lys residue (K),
comprises in position 6 of SEQ ID NO: 2 a Lys residue (K), and in position 49 of SEQ ID NO: 2 an Asp residue (D),
comprises in position 7 of SEQ ID NO: 2 a Gly residue (G), and in position 8 of SEQ ID NO: 2 an Ile residue (I), and in position 9 of SEQ ID NO: 2 a Gly residue (G),
comprises in position 3 of SEQ ID NO: 2 an Arg residue (R), in position 6 of SEQ ID NO: 2 a Lys residue (K), in position 7 of SEQ ID NO: 2 a Gly residue (G), in position 8 of SEQ ID NO: 2 an Ile residue (I), in position 9 of SEQ ID NO: 2 a Gly residue (G) and in position 49 of SEQ ID NO: 2 an Asp residue (D).

Positions 6, 49, 7, 8 and 9 determined in relation to SEQ ID NO: 2 correspond respectively to positions 263, 306, 264, 265 and 266 of human Annexin A3, identified as SEQ ID NO: 5, which corresponds to the amino acid sequence of complete native human Annexin A3 (UniProtKB accession No. P12429).

Preferably, the antibody directed against the first repeat domain of native human Annexin A3, the sequence of which is identified as SEQ ID NO: 1, is the capture antibody, and the antibody directed against the fourth repeat domain of native human Annexin A3, the sequence of which is identified as SEQ ID NO: 2, is the detection antibody.

The antibodies specific to the first repeat domain of native human Annexin A3, and specific to the fourth repeat domain of native human Annexin A3, i.e. the capture and detection antibodies described above, are antibodies with a high affinity with an affinity constant of at least 10⁻⁹, preferably at least 10⁻¹⁰, and which furthermore have a low dissociation constant, of less than 2 10⁻³ s⁻¹, more preferably less than 5 10⁻⁴ s⁻¹.

Preferably, the antibodies are monoclonal antibodies, and the preferred antibodies are the following antibodies: TGC42, TGC43 and TGC44 used as capture antibodies, with 13A12G4H2 and 1F10A6 used as detection antibodies. The preferred pair comprising capture antibody TGC44, and detection antibody 13A12G4H2.

Another object of the invention is an immunoassay kit for in vitro diagnosis of a prostate cancer in a urine sample to be analysed, comprising two antibodies; a capture antibody and a detection antibody, one of the two antibodies being directed against the first repeat domain of native human Annexin A3, the sequence of which is identified as SEQ ID NO: 1, and the other antibody being directed against the fourth repeat domain of native human Annexin A3, the sequence of which is identified as SEQ ID NO: 2 and an explanatory note.

Preferably, the capture antibody is directed against the first repeat domain of native human Annexin A3, the sequence of which is identified as SEQ ID NO: 1, and the detection antibody is directed against the fourth repeat domain of native human Annexin A3, the sequence of which is identified as SEQ ID NO: 2.

The capture and detection antibodies in the kit have the same characteristics as those described above for the process.

The term antibody refers to a polyclonal antibody, a monoclonal antibody, a humanised antibody, a human antibody or a fragment of said antibodies, in particular fragments Fab, Fab′, F(ab′)2, ScFv, Fv, Fd. The requisite condition is that said antibodies must be specific to Annexin A3, i.e. that they do not have any cross reactions with other annexins, and that they are specific to the first repeat domain of Annexin A3, or specific to the fourth repeat domain of Annexin A3; with the antibodies with the highest affinities and lowest dissociation constants being the preferred antibodies.

Polyclonal antibodies can be obtained by immunising an animal with the appropriate immunogen, followed by retrieval of the target antibodies in purified form, by sampling serum from said animal, and separating said antibodies from the other constituents of the serum, in particular by affinity chromatography on a column, on which is bound an antigen specifically recognised by the antibodies.

Monoclonal antibodies can be obtained by the hybridomas technique, the general principle of which is reiterated below.

Initially, an animal, generally a mouse, is immunised with the appropriate immunogen, the B lymphocytes of which are capable of producing antibodies against this antigen. These antibody-producing lymphocytes are then fused with “immortal” myelomatous cells (of mice in this example), in order to produce hybridomas. From the heterogeneous mixture of the cells obtained in this way, we make a selection of cells capable of producing a particular antibody and reproducing indefinitely. Each hybridoma is reproduced in clone form, each leading to the production of a monoclonal antibody whose recognition properties with regard to the protein can be tested for instance by ELISA, by one or two-dimension immunotransfer (Western blot), by immunofluorescence, or using a biosensor. The monoclonal antibodies selected in this way are then purified, in particular according to the affinity chromatography technique described above.

The monoclonal antibodies may also be recombinant antibodies obtained by genetic engineering, using techniques well known to the person skilled in the art.

The capture antibody is preferably bound, directly or indirectly, onto a solid support, e.g. a cone or a microtitration plate well, etc. . . .

The detection antibody is labelled using a label reagent capable of directly or indirectly generating a detectable signal. A non-exhaustive list of these label reagents consists of:
enzymes producing a signal detectable for example by colorimetry, fluorescence, luminescence, such as horseradish peroxidase, alkaline phosphatase, β-galactosidase, or glucose-6-phosphate dehydrogenase,
chromophores such as fluorescent or luminescent compounds, or dyes,
fluorescent molecules such as Alexas or phycocyanins,
radioactive molecules such as ³²P, ³⁵S or ¹²⁵I.

Indirect detection systems can also be used, such as for example ligands capable of reacting with an anti-ligand. The ligand/anti-ligand pairs are well known to the person skilled in the art, which is the case for instance with the pairs below: biotin/streptavidin, hapten/antibody, antigen/antibody, peptide/antibody, sugar/lectin. In this case, it is the ligand which carries the binding partner. The anti-ligand may be detectable directly by the label reagents described in the paragraph above, or itself be detectable by a ligand/anti-ligand.

These indirect detection systems may, under certain conditions, lead to signal amplification. This signal amplification technique is well known to the person skilled in the art, and reference may be made to the Applicant's prior patent applications FR98/10084 or WO-A-95/08000 by the Applicant.

According to the type of labelling used, the person skilled in the art will add reagents to make the labelling visible.

The process according to the invention is a “sandwich”-type immunoassay carried out on a urine sample, in particular an “expressed” urine sample, i.e. a urine sample obtained after a digital rectal examination.

The invention will be better understood using the examples below, provided as an illustrative and non-exhaustive guide, and also using the appended figures.

### FIGURES

FIG. 1 corresponds to graphs relating to ELISA assay of Annexin A3 in ng/mL, in urines expressed after digital rectal examination, in 6 patients (Patients A to F), assayed individually with each ELISA sandwich assay format, the name of which is indicated on the X-axis. The numerical values represented on this graph are presented in Table 4;

FIG. 2 shows the ELISA assay of Annexin A3 in analysis by successive filtrations (on a 0.45 μm filter, then 0.22 μm and then 0.02 μm) of eight urines expressed after digital rectal examination. The analysis was performed within 3-4 hours of collecting the sample. 5C5B10 assay panel: the samples and their fractions were assayed using TGC44/5C5B10 ELISA. 13A12G4H2 assay panel: the samples and their fractions were assayed using TGC44/13A12G4H2 ELISA. NF: non filtered, control; FT: filtrate, indicating the cut-off threshold of the filter used. For each group of 8 fractions, the horizontal line represents the mean of the 8 measurements observed. The doses of Annexin A3 are expressed as % of the initial dose;

FIG. 3 shows the ELISA assay of Annexin A3 in analysis by successive centrifuging (800 g, then 12,000 g and 150,000 g) of eight urines expressed after digital rectal examination. The analysis was performed within 3-4 hours of collecting the sample. 5C5B10 assay panel: the samples and their fractions were assayed using TGC44/5C5B10 ELISA. 13A12G4H2 assay panel: the samples and their fractions were assayed using TGC44/13A12G4H2 ELISA. NC: non-centrifuged, control; SN: supernatant with the indicated centrifuging speed. For each group of 8 fractions, the horizontal line represents the mean of the 8 measurements observed. The doses of Annexin A3 are expressed as % of the initial dose;

FIG. 4 shows the ELISA assay of Annexin A3 in analysis by successive centrifuging (800 g, 12,000 g and 150,000 g) of ten other urines expressed after the digital rectal examination. The analysis was performed within 3-4 hours of collecting the sample. 5C5B10 assay panel: the samples and their fractions were assayed using TGC44/5C5B10 ELISA. 13A12G4H2 assay panel: the samples and their fractions were assayed using TGC44/13A12G4H2 ELISA. % soluble=dose in ultracentrifuging supernatant/initial dose×100; % exosome=(supernatant 12,000 g/initial dose×100)−% soluble; % membrane=100−(supernatant 12,000 g/initial dose×100).

FIG. 5 shows the reactivity of the 4 repeat domains D1 to D4 of Annexin A3, expressed in a recombinant form, with the 6 monoclonal anti-Annexin A3 antibodies indicated and analysed by the Western blot technique. For gels TGC42 and 1F10A6, the first well corresponds to the molecular weight marker;

FIG. 6 is an alignment of the sequences of the various recombinant proteins expressing domains of Annexin A3, and summarises the immunoreactivity of each of these recombinants with the monoclonal TGC44 antibody, analysed by the Western blot technique. The three suspension points at the N-terminus or C-terminus end of a sequence indicate that it continues in the construction, although it is not fully shown in the figure. The stop codon is shown by a star (*);

FIG. 7 illustrates the impact of the Alanine mutations of domain D4 on the recognition of ANXA3, using antibodies 13A12G4H2 and 1F10A6. The analysis was carried out using the ELISA technique. The capture antibody is TGC44. Antibodies 13A12G4H2 and 1F10A6 tested were used for detection. The position of the Alanine mutations in the protein sequence of ANXA3 is shown on the X-axis. The Y-axis is the “signal fold change”, which corresponds to log₂(mutated protein signal/non-mutated protein signal). Antibody 5C5B10, which is not directed against domain D4, is not affected, it is used as a control. The arrows show the mutations for which there is disruption of the recognition signal for 13A12G4H2 and 1F10A6, which makes it possible to define the residues involved in the binding of these antibodies.

FIG. 8 shows the absence of cross reactivity of the ELISA assay formats described with 7 other proteins in the annexins family. The graph shows the ELISA signal obtained in “Relative Fluorescence Values” (RFV) on the VIDAS® device for each of the test formats TGC44/5C5B10 and TGC44/13A12G4H2, and for each annexin indicated on the X-axis. By way of comparison, under the experimental conditions used, Annexin A3 was able to obtain a signal of more than 6000 RFV with each test format;

FIG. 9 shows the sensorgrams obtained for each of the 6 monoclonal antibodies characterised using Biacore™ T100. The graphs show the resonance signal measured in “Resonance Units” (RU) as a function of time. Each graph curve shows all the measurements taken for a given Annexin A3 concentration. For each antibody, 9 dilutions between 0 and 64 nM of Annexin A3 were analysed, and are shown;

FIG. 10 brings together graphs for the ELISA assay of Annexin A3 in urines expressed after digital rectal examination, in patients with prostate cancer confirmed by biopsy (cancerous), and patients with a prostate pathology, but where malignity has been ruled out (non-cancerous). The “non-cancerous” group primarily comprises patients with benign prostatic hypertrophy. The graphs on the left of the page entitled 5C5B10_SG show the dose of Annexin A3 measured by the VIDAS TGC44/5C5B10 assay in ng/mL, and standardised by urinary density measured by the Combur™ 10 strip, using the formula: standardised dose=VIDAS dose/(urinary density−1). Similarly, the graphs on the right of the page entitled 13A12G4H2_SG show the dose of Annexin A3 measured by the VIDAS TGC44/13A12G4H2 assay in ng/mL, and standardised by urinary density. Two different patient cohorts were studied: cohort #1 includes 127 patients, and cohort #2 94 patients. For each series of data, the mean of the series is shown in the form of a horizontal line on the graphs. The probability associated with the unilateral Mann-Whitney test is also indicated on each graph: p-value<0.05=*, p-value<0.01=**, ns=not significant.

FIG. 11 shows the effect of the calcium ion and chelating agent EDTA on doses measured with the 5C5B10 and 13A12G4H2 assays. Eleven urines collected after digital rectal examination were assayed directly (without treatment) or after addition of 5 or 25 mM of CaCl₂ or after addition of 5 or 25 mM of EDTA. The Y-axis shows the ratio of doses with treatment (Ca₂₊ or EDTA)/dose without treatment. The median of the series is represented in the form of a horizontal line on the graphs. The probability associated with the bilateral Wilcoxon signed-rank test used to compare 1 ratio to 1, this probability having been corrected for multiple Bonferroni tests, is also indicated for each series.

### EXAMPLES

### Example 1

### Detecting Annexin A3 by ELISA in Different Types of Sample

Obtaining Monoclonal Antibodies

Immunisation trials were performed on female BALB/c (H-2^{d}) mice aged from six to eight weeks at the time of the first immunisation. The native human Annexin A3 protein was purchased from the company Arodia Arotech Diagnostic (Cat No. 25592), it is purified from human neutrophils. This protein was mixed volume for volume with Freund's adjuvant (Sigma), prepared in the form of a water-in-oil emulsion. It is known to possess a good immunogenic capacity. They received three successive doses of 10 μg of immunogen, at zero, two and four weeks. All the injections are made subcutaneously. The first injection is made in mixture with complete Freund's adjuvant, and the following two are made in mixture with incomplete Freund's adjuvant. Between D50 and D70 after the first injection, the humoral responses were restimulated with an intravenous injection of 100 μg of native protein.

In order to monitor the appearance of antibodies, blood samples are regularly taken from the mice. The presence of anti-ANXA3 antibodies is tested using an ELISA. The protein of interest is used for capture (1 μg/well), after saturation various dilutions of serums to be tested are reacted with the antigen (incubation at 37° C., for 1 h). The specific antibodies present in the serum are revealed by an AffiniPure goat anti-mouse IgG antibody conjugated using alkaline phosphatase (H+L, Jackson Immunoresearch, Cat No. 115-055-146), which binds to the target antibodies (0.1 μg/well).

Three days after the last injection, one of the immunised mice was sacrificed; the blood and spleen were sampled. The splenocytes obtained from the spleen were cultured with the Sp2/0-Ag14 myeloma cells in order to fuse and immortalise, according to the protocol described by (8, 9). After an incubation period of 12-14 days, the hybridoma supernatants obtained were screened to determine the presence of anti-ANXA3 antibodies using the ELISA test described in the paragraph above. The immunogen (native ANXA3), recombinant Annexin A3 produced in E. coli, and various human cells expressing ANXA3 were applied in succession in order to screen the hybridoma supernatants. The positive hybridoma colonies were sub-cloned twice according to the limit dilution technique, well known to the person skilled in the art.

In this way the anti-annexin A3 monoclonal antibodies 5C5B10, 13A12G4H2, 9C6B4, 6D9D10, and 1F10A6 were obtained.

Selecting Anti-ANXA3 Monoclonal Antibodies for Immunoassay of ANXA3

The complementarity of the various anti-ANXA3 antibodies obtained as described above, and of antibodies TGC42, TGC43 and TGC44 described in patent application WO 2010/034825, was analysed using native ANXA3 as the antigen (immunogen), diluted in PBS buffer, by performing a sandwich immunoassay. This type of assay can be performed on a microplate, automatically or manually, or also using automated immunoanalysers such as VIDAS (bioMérieux).

We used Vidas® HBs Ag Ultra kit reagents (bioMérieux, Cat No. 30315), as described in the corresponding manual (ref. 11728 D-FR-2005/5) and modified in this way:
Cones were sensitised with one of the capture antibodies to be tested, TGC42, TGC43, TGC44 or 9C6B4, at a concentration of 10 μg/mL.
The content of the second well of the HBs Ag Ultra cartridge was replaced by 300 μL of detecting antibody to be tested (5C5B10, 13A12G4H2, 9C6B4, 6D9D10, 1F10A6, TGC42, TGC43, TGC44), coupled to biotin, diluted to 1 μg/mL in the buffer of the second well of the Vidas® HBs Ag Ultra kit (well X1), containing goat serum and 1 g/L sodium azide.
Native ANXA3 protein is tested diluted to 100, 25 and 3 ng/mL in PBS buffer. The sample is deposited (150 μL) into the first well (well X0) of the HBs Ag Ultra cartridge.
The ELISA reaction was performed using the Vidas® automated machine and the protocol described for the HBs Ag Ultra test.
The results were obtained in the form of raw values after subtracting background noise. The signal is in RFV (relative fluorescent value).

Table 1 below summarises the results obtained (RFV signal), with the various combinations of antibodies used for capture or detection, for three dilutions of purified neutrophil-derived native annexin A3 (100, 25 and 3 ng/mL).

TABLE 1

| Biotinylated detection antibodies | | | | | |
|---|---|---|---|---|---|
| | ANXA3 dilution | Capture antibodies | | | |
| Clone | ng/ml | TGC42 | TGC43 | TGC44 | 9C6B4 |
| TGC42 | 0 | — | 22 | 11 | 17 |
| | 3 | — | 44 | 20 | 78 |
| | 25 | — | 238 | 89 | 500 |
| | 100 | — | 859 | 297 | 1837 |
| TGC43 | 0 | 106 | — | 39 | 63 |
| | 3 | 111 | — | 36 | 82 |
| | 25 | 144 | — | 56 | 257 |
| | 100 | 232 | — | 86 | 914 |
| TGC44 | 0 | 21 | 9 | — | 12 |
| | 3 | 32 | 19 | — | 19 |
| | 25 | 98 | 89 | — | 74 |
| | 100 | 303 | 309 | — | 261 |
| 9C6B4 | 0 | 51 | 42 | 33 | — |
| | 3 | 65 | 122 | 39 | — |
| | 25 | 201 | 744 | 117 | — |
| | 100 | 590 | 3525 | 357 | — |
| 5C5B10 | 0 | 20 | 12 | 93 | 9 |
| | 3 | 5244 | 3669 | 5870 | 39 |
| | 25 | 10480 | 9874 | 10429 | 249 |
| | 100 | 11310 | 11095 | 11168 | 1139 |
| 13A12G4H2 | 0 | 26 | 21 | 18 | 19 |
| | 3 | 2298 | 676 | 3733 | 23 |
| | 25 | 9655 | 8468 | 10208 | 21 |
| | 100 | 11380 | 10995 | 11332 | 21 |
| 6D9D10 | 0 | 12 | 23 | 16 | 9 |
| | 3 | 11 | 23 | 14 | 9 |
| | 25 | 12 | 23 | 18 | 19 |
| | 100 | 18 | 41 | 43 | 27 |
| 1F10A6 | 0 | 45 | 9 | 14 | 14 |
| | 3 | 159 | 207 | 194 | 17 |
| | 25 | 4014 | 6728 | 5680 | 13 |
| | 100 | 10489 | 10931 | 10688 | 15 |

Table 2 below summarises the results obtained described in Table 1, but with a different reading grid, to facilitate analysis and interpretation:

If RFV at 3 ng/mL<1000 then “−”

If RFV at 3 ng/mL>1000 then “+”

If RFV at 25 ng/mL>3000 then “++”

If (RFV at 100 ng/mL>9000) and (3000<RFV at 25 ng/mL<9000) then “+++”

If RFV at 100 ng/mL and 25 ng/mL>9000 then “++++”

TABLE 2

| capture | biotinylated detection mAb* | | | | | | | |
|---|---|---|---|---|---|---|---|---|
| mAb* | TGC42 | TGC43 | TGC44 | 9C6B4 | 5C5B10 | 13A12G4H2 | 1F10A6 | 6D9D10 |
| TGC42 | − | − | − | − | ++++ | ++++ | +++ | − |
| TGC43 | − | − | − | + | ++++ | +++ | +++ | − |
| TGC44 | − | − | − | − | ++++ | ++++ | +++ | − |
| 9C6B4 | + | − | − | − | + | − | − | − |

mAb = monoclonal antibody
As the table above shows, there are 9 combinations of complementary monoclonal antibodies which enable sandwich ELISA assaying of ANXA3, with a highly satisfactory signal dynamic (“+++” and “++++” pairs). Other combinations of complementary monoclonal antibodies are possible, namely 9C6B4/TGC42, 9C6B4/TGC43 and 9C6B4/5C5B10, but these solutions are not sufficiently robust from an analytical viewpoint, and do not have sufficient analytical sensitivity to be used in urine.

For capture, monoclonal antibodies TGC42, TGC43 and TGC44 have equivalent performances with regard to Annexin 3.

For detection, monoclonal antibodies 5C5B10 and 13A12G4H2 also have equivalent performance to Annexin 3, while monoclonal antibody 1F10A6 provides satisfactory signals, but weaker than with monoclonal antibodies 5C5B10 and 13A12G4H2.

Selecting Anti-ANXA3 Monoclonal Antibodies According to their Ability to Detect Prostatic ANXA3

The complementarity and ability of anti-ANXA3 antibodies to detect prostatic ANXA3 was analysed using the ELISPOT technique. This is a variant of the ELISA technique which directly detects cultured cell secretions. The WPE1-NB26 human cells (ATCC Cat No. CRL-2852), derived from a prostatic epithelial line RWPE-1 (ATCC Cat No. CRL-11609), were chosen as the expression model of human ANXA3 produced by the prostate. These cells were transformed into cancerous cells by MNU treatment (N-methyl-N-nitrosourea) (10). They are cultured in K-SFM medium (Gibco), supplemented with 5 ng/mL of EGF (Epidermal Growth Factor) and 0.05 mg/mL of BPE (Bovine Pituitary Extract). The lines are incubated at 37° C. with 5% CO₂.

The monoclonal capture antibodies (TGC42, TGC43, TGC44, 5C5B10, 13A12G4H2, 9C6D9, and 1F10A6) were adsorbed on MultiScreen™ HTS 96-well plates (Millipore, Cat No. MSIP4510), at a concentration of 1 μg/well in sterile PBS for one night at 4° C. The plates are then washed in PBS and saturated with culture medium containing 10% foetal calf serum (FCS). In parallel, the cells are counted, and then diluted and distributed with 1000 cells per well. The plates are incubated for 20 h at 37° C. and 5% CO₂, and then emptied. The remaining cells are then lysed by treatment in chilled water for 10 minutes. The plates are then washed with PBS containing 0.05% Tween-20. The biotinylated detection antibodies (TGC42, TGC43, TGC44, 5C5B10, 13A12G4H2, 9C6D9 and 1F10A6) are added at 0.1 μg/well diluted in PBS-1% BSA-0.05% Tween-20, and incubated for 2 h at ambient temperature. After several washes, the spots are revealed by adding extravidin-alkaline phosphatase (Sigma, Cat No. E2636) for one hour at ambient temperature, followed by the substrate 5 bromo-4-chloro-3-indolyl phosphate/nitro blue tetrazolium (BCIP/NBT, Biorad, Cat No. 170-6432).

The secretion of Annexin A3 by WPE1-NB26 cells is measured qualitatively by the observer. The number of spots observed is graded on a scale of − to ++++ (Table 3). The best solution for detecting Annexin A3 of prostatic origin is to pair an antibody from TGC42, TGC43 or TGC44 with an antibody chosen from 13A12G4H2 and 1F10A6. The chosen antibodies can equally be used either for capture or detection, the only condition being that there is an associated antibody from each group.

The results are summarised in Table 3 below.

TABLE 3

| capture | biotinylated detection mAb* | | | | | | |
|---|---|---|---|---|---|---|---|
| mAab* | TGC42 | TGC43 | TGC44 | 9C6B4 | 5C5B10 | 13A12G4H2 | 1F10A6 |
| TGC42 | − | − | − | + | ++ | ++++ | ++++ |
| TGC43 | − | − | − | + | ++ | ++++ | ++++ |
| TGC44 | − | − | − | + | ++ | ++++ | ++++ |
| 9C6B4 | − | − | − | − | − | − | − |
| 5C5B10 | − | − | − | + | − | − | − |
| 13A12G4H2 | ++++ | ++++ | ++++ | + | − | − | − |
| 1F10A6 | +++ | ++++ | +++ | + | − | − | − |

*mAab = monoclonal antibody
Very surprisingly, the annexin A3 secreted by a cancerous prostatic line is less well detected when there is an associated antibody chosen from TGC42, TGC43 and TGC44 for capture, with antibody 5C5B10 for detection. Now, with the VIDAS ® immunoassay performed on purified neutrophil-derived annexin A3, such a difference between antibodies 5C5B10 and 13A12G4H2 was not observed, as shown in Table 2.

Detecting Annexin A3 in Urines Expressed from Patients after Digital Rectal Examination

Six urine samples expressed after digital rectal examination were obtained and treated according to the process described in patent application WO2007/141043 and by (11). The mean prostate palpation time was 10 seconds, rather than the 20 seconds indicated in application WO2007/141043.

The ANXA3 contained in these various biological samples was quantified with each of the nine ELISA assays selected. The biotinylated detection antibodies were diluted to 0.5 μg/mL for the monoclonals 5C5B10 and 13A12G4H2, and to 1 μg/mL for the clone 1F10A6. For each ELISA assay, a calibration curve was determined by assaying a concentration range of the purified native Annexin A3 protein (Arodia). The calibration curve was plotted, with on the X-axis the base-ten logarithm of the concentration, and on the Y-axis the signal measured by Vidas® in RFV. The concentration of ANXA3 present in the biological sample was calculated by interpolating the concentration corresponding to the RFV signal read by the Vidas®, using non-linear regression mathematical models, such as a third-order polynomial or a 4-PL model, well known to the person skilled in the art. The results are set out in Table 4 below.

TABLE 4

| | Doses of annexin A3 in ng/mL according to the ELISA assay format | | | | | | | | |
|---|---|---|---|---|---|---|---|---|---|
| | (Capture antibodies - Detection antibodies) | | | | | | | | |
| | TGC42- | TGC43- | TGC44- | TGC42- | TGC43- | TGC44- | TGC42- | TGC43- | TGC44- |
| P* | 5C5B10 | 5C5B10 | 5C5B10 | 13A12G4H2 | 13A12G4H2 | 13A12G4H2 | 1F10A6 | 1F10A6 | 1F10A6 |
| A | 90 | 99 | 123 | 0.1 | 0.1 | 0.1 | 0.1 | 0.1 | 0.1 |
| B | 5 | 2 | 5 | 9 | 11 | 13 | 13 | 14 | 14 |
| C | 2 | 1 | 2 | 6 | 8 | 8 | 9 | 9 | 10 |
| D | 11 | 3 | 13 | 78 | 80 | 81 | 77 | 86 | 97 |
| E | 22 | 7 | 28 | 110 | 144 | 154 | 120 | 136 | 149 |
| F | 1100 | 810 | 2100 | 0.0 | 0.0 | 0.0 | 0.0 | 0.0 | 0.0 |

P* = patient

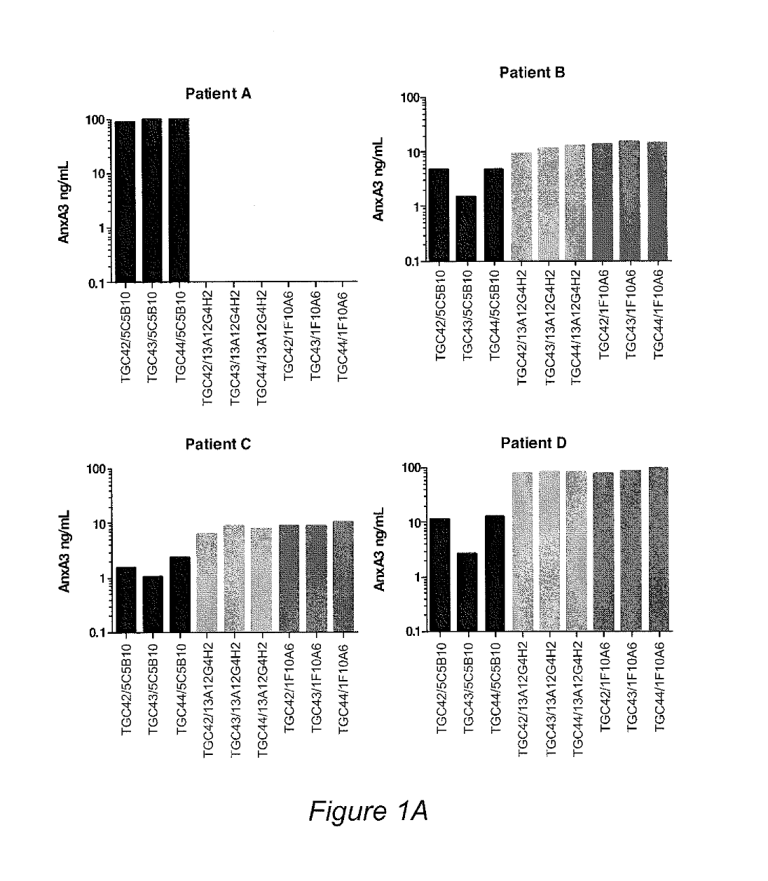

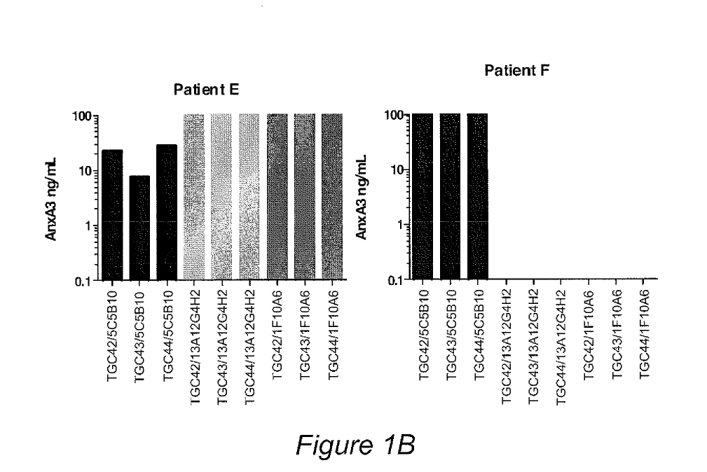

The results of the Vidas® ELISA assays of urinary ANXA3 in the six patients are summarised in Table 4, and represented in FIG. 1. These results are extremely surprising, and reveal a previously unsuspected complexity of urinary ANXA3. Indeed, for a given sample, the 9 ELISA test formats do not always yield the same dose. This difference is associated with the detection antibody: for a given sample and a given detection antibody, the doses yielded are very similar, or even identical, regardless of the capture antibody (TGC42, TGC43 or TGC44). The correlation between the doses obtained by the formats using TGC42 and those obtained by the formats using TGC43 is 0.956 (Spearman r, p-value<0.0001). The correlation between the doses measured by the formats using TGC42 and that obtained by the formats using TGC44 is 0.994 (Spearman r, p-value<0.0001). The 3 capture antibodies exhibit very similar behaviour. They are not the cause of the differences observed.

The concentrations of Annexin A3 calculated with ELISAs using antibodies 13A12G4H2 or 1F10A6 for detection are also correlated (Spearman r=0.999, p-value<0.0001). The gradient of the straight line calculated, with on the X-axis the doses obtained by ELISAs using 13A12G4H2 and on the Y-axis those obtained by ELISAs using 1F10A6 is 1: these two antibodies yield identical doses. Conversely, there is no correlation between the doses calculated with antibody 5C5B10 used for detection and those calculated with 13A12G4H2.

According to this analysis performed on expressed urines, the 9 ELISA assays can be divided into 2 groups. Group 1 corresponds to the combinations of capture antibodies TGC42, TGC43 or TGC44, with clones 13A12G4H2 or 1F10A6 used for detection. Group 2 corresponds to the combinations of the capture antibodies TGC42, TGC43 or TGC44 with monoclonal 5C5B10 used for detection. As each of these antibodies is highly specific to ANXA3, the two ELISA groups measure a different piece of biological information. The ELISPOT analysis presented above suggests that the group 1 ELISAs using clones 13A12G4H2 or 1F10A6 are more suitable for detecting ANXA3 of prostatic origin.

### Example 2

### Characterising Urinary Annexin A3

Eight urine samples expressed post-digital rectal examination were obtained according to the process described in example 1, transported from the hospital to the laboratory and submitted to the analyses described within 4 h of collection. The TGC44/13A12G4H2 assay (hereafter called simply 13A12G4H2) was chosen as the prototype assay for group 1 defined in example 1. Similarly, the TGC44/5C5B10 assay (hereafter called simply 5C5B 10) was chosen as the prototype assay representing group 2.

Analysis of Expressed Urine by Successive Filtrations

The freshly collected urines underwent successive filtrations:
A first filtration on a membrane with pores 0.45 μm in diameter (Millex), after which the filtrate is recovered and an aliquot set aside for assays: FT 0.45 μm.
The FT 0.45 μm filtrate is filtered on a 0.22 μm membrane (Millex) and an aliquot set aside for assays: FT 0.22 μm.
The FT 0.22 μm filtrate is filtered again, this time on a 0.02 μm membrane (Millipore), the pore size of which retains urinary exosomes, i.e. FT 0.02 μm.

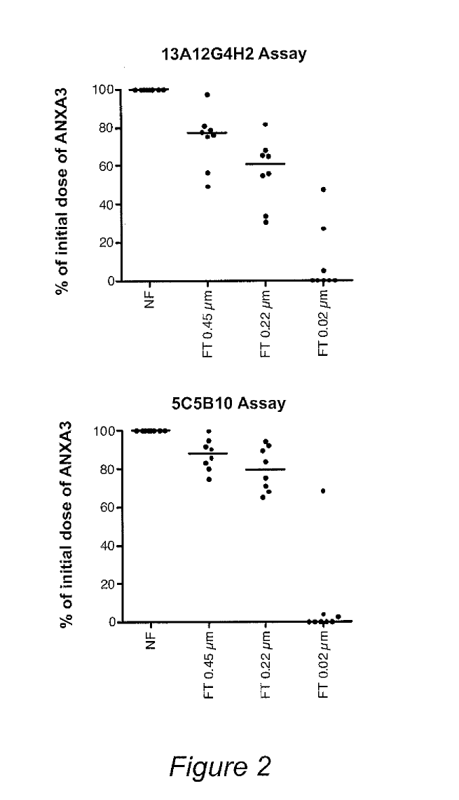

Each of these fractions is then assayed with both prototype ELISA test formats on a Vidas® automated machine. The Annexin A3 doses measured in each fraction are expressed as a percentage of the initial dose of urinary Annexin A3 before initial filtration (=100%). The results are shown in FIG. 2. The reduction in the dose between two successive filtrates indicates that biological particles containing ANXA3 were retained on the filtration membrane. Hence according to principles well known to the person skilled in the art, a filtration on 0.45 μm retains the cells and the cellular debris (12, 13), a filtration on 0.22 μm retains certain organelles and subcellular fractions and a filtration on 0.02 μm retains exosomes (14-16). This reduction is properly observed for all the samples, with both assay processes used (5C5B10 and 13A12G4H2). In the vast majority of the urines expressed (6/8), approximately 20 to 40% of the ANXA3 is associated with cellular debris or other subcellular fractions. The remaining 60 to 80% are eliminated by filtration on 0.02 μm, and are therefore associated with exosomes. Of the 8 expressed urine samples tested, soluble ANXA3 (which passes through a 0.02 μm filter) was found only in a single sample with the 5C5B10 assay, and in two samples with the 13A12G4H2 assay.

Analysis of Expressed Urine by Successive Centrifuging

The same freshly collected urines were also fractionated by successive centrifuging:
The urines are centrifuged the first time at 800 g for 5 min, the supernatant is recovered and an aliquot set aside for assays: SN 800.
The urinary supernatant derived from centrifuging at 800 g is centrifuged at 12,000 g for 7 min, the supernatant is recovered and an aliquot set aside for assays: SN 12,000.
The urinary supernatant obtained after centrifuging at 12,000 g is then ultracentrifuged at 150,000 g overnight at 4° C. in order to precipitate the urinary exosomes in the pellet. The supernatant collected is SN ultra.

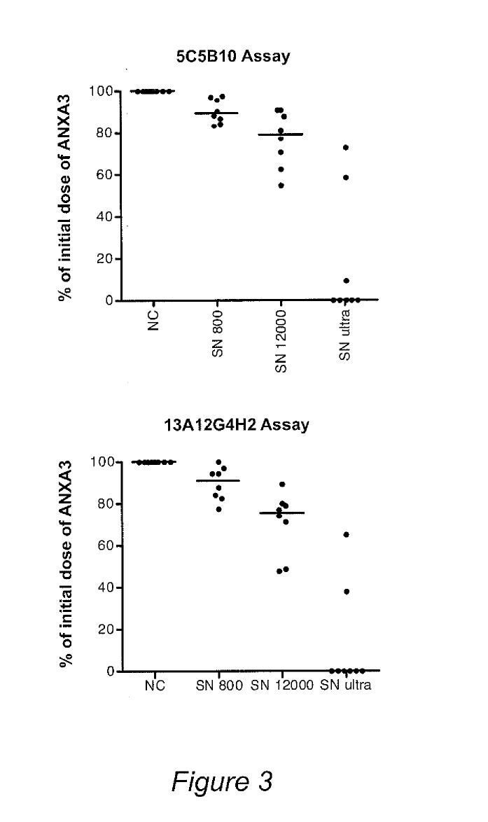

Each of these fractions is then assayed with both prototype ELISA test formats on a Vidas® machine. The Annexin A3 doses measured in each fraction are expressed as a percentage of the initial dose of urinary Annexin A3 before any centrifuging (NC=100%). The results are shown in FIG. 3. The reduction in the dose between two successive supernatants indicates that biological particles containing ANXA3 were separated into the pellet during centrifuging. Hence according to principles well known to the person skilled in the art, centrifuging at 800 g aggregates most human cells, centrifuging at 12,000 g aggregates residual cellular debris and certain organelles, and ultracentrifuging overnight aggregates all the subcellular particles, including exosome type vesicles (17, 18). Any protein remaining in the supernatant after an ultracentrifuging is considered to be soluble (19). This reduction is properly observed for all the samples, with both assay processes used (5C5B10 and 13A12G4H2). In the vast majority of the urines expressed (6/8), approximately 20 to 25% of the ANXA3 is associated with cellular debris or other subcellular fractions. The remaining 75 to 80% are only precipitated by ultracentrifuging overnight, and are therefore associated with exosome type particles. Of the 8 expressed urine samples tested, soluble ANXA3 (which remains in the supernatant after one night of ultracentrifuging) was found only in the two samples already identified as containing soluble ANXA3 with the analysis set out in paragraph 1, and this with both assay techniques 5C5B10 and 13A12G4H2.

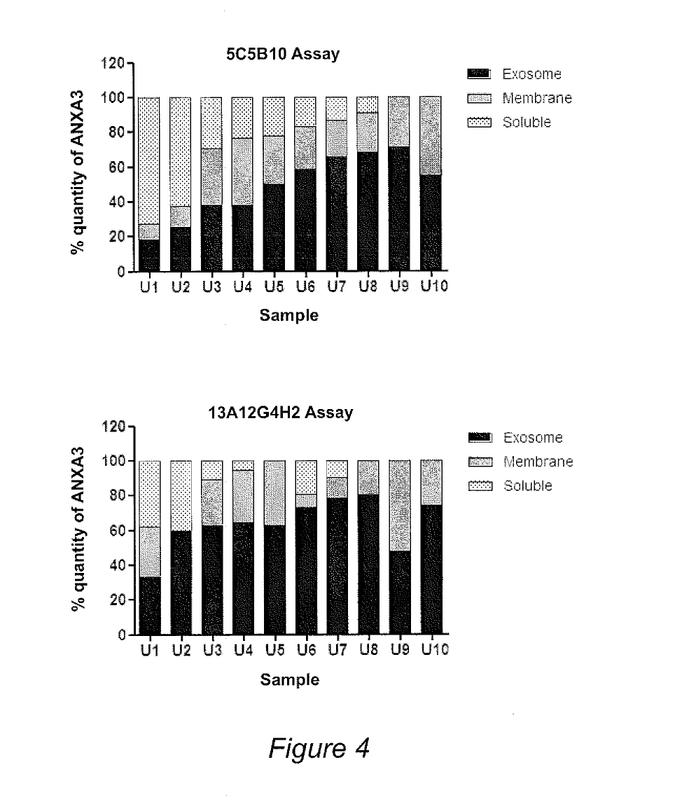

A new series of 10 freshly collected urines was analysed, under slightly modified centrifuging conditions. The 800 g centrifuging was 10 min, and the 12,000 g centrifuging was 30 min. The results of this second experiment of successive centrifuging are set out in FIG. 4, and confirm the observations made in the first experiment (FIG. 3). Furthermore, they show that the 13A12G4H2 assay recognises ANXA3 associated with exosomes more frequently than the 5C5B10 assay.

Overall the results of fractionation by filtration and by centrifuging are extremely closely matched, and indicate that most of the ANXA3 to be assayed is associated with exosomes, but not exclusively. We demonstrated that ANXA3 is also associated with larger-sized particles such as cellular debris, and that it could also be in soluble form. The sample preparation for a Western blot analysis makes it possible to solubilise and denature the proteins, and thus reduce this complexity, which can partly explain the difficulties encountered hitherto in finding an ELISA assay process which is usable in biological fluids, and in particular urine. Of course, the treatment process of the expressed urines collected and the storage conditions (temperature, buffer), are also factors which can cause variation of the distribution of ANXA3 in the different urinary fractions in which it may be present.

### Example 3

### Determining the Epitopes Recognised by Anti-ANXA3 Monoclonal Antibodies

Expression of 4 “Annexin Repeat” Domains of Annexin A3 in Recombinant Form, and Determining the Repeat Domains Recognised by Monoclonal Antibodies.

Like all members of the Annexins family, Annexin A3 contains in its protein sequence so-called “annexin repeat” domains. There are 4 of these repeat domains, which characterise the family. In order to determine the repeat domain recognised by each monoclonal antibody, these domains were expressed in a recombinant form. A sequence of 8 histidines was added to the N-terminus part of each domain, in order to enable purification by metal-chelate affinity chromatography. Table 5 summarises the protein sequences of the recombinant constructions permitting expression of each of the repeat domains in isolation.

TABLE 5

| Domain | Actual ^{a} | Expressed ^{b} | Protein sequence |
|---|---|---|---|
| D1 | 27-87 | 19-89 | MGHHHHHHHHSPSVDAEAIQKAIRGIGTDEKMLISI |
| | | | LTERSNAQRQLIVKEYQAAYGKELKDDLKGDLSGH |
| | | | FEHLMVALVT |
| | | | (SEQ ID: 6) |
| D2 | 99-159 | 92-160 | MGHHHHHHHHAVFDAKQLKKSMKGAGTNEDALIE |
| | | | ILTTRTSRQMKDISQAYYTVYKKSLGDDISSETSGDF |
| | | | RKALLTLA |
| | | | (SEQ ID: 7) |
| D3 | 183-243 | 171-245 | MGHHHHHHHHDEHLAKQDAQILYKAGENRWGTD |
| | | | EDKFTEILCLRSFPQLKLTFDEYRNISQKDIVDSIKGE |
| | | | LSGHFEDLLLAIVN (SEQ ID: 8) |
| D4 | 258-318 | 252-323 | MGHHHHHHHHAFLAERLHRALKGIGTDEFTLNRIM |
| | | | VSRSEIDLLDIRTEFKKHYGYSLYSAIKSDTSGDYEIT |
| | | | LLKICGGDD |
| | | | (SEQ ID: 9) |

^{a} Actual: Repeat domain length, from the first to last amino acid, according to the UniProtKB database (http://www.uniprot.org).
^{b} Expressed: Length of construction containing the repeat domain, amino acid numbering according to UniProtKB. The constructions contain some additional amino acids at the N and C-termini of the domain, so as not to interrupt the alpha helices and enable them to form.

The nucleic acid sequences corresponding to the protein sequences of domains D D2, D3 and D4 were obtained by chemical synthesis by the company Geneart. These nucleic sequences were optimised to promote expression of the proteins in Escherichia coli. The DNA fragments were cloned between sites Nco I and Xba I of the procaryote expression vector pMRCH79 (derivative of pMR78, bioMérieux). The plasmids obtained in this way were transformed in bacteria BL21 (DE3) (Stratagene). The cultures for producing the various domains are performed at 37° C., under stirring, in 2-YT medium (Invitrogen). Induction is performed with 0.5 mM of IPTG (isopropyl beta-1-thiogalactosidase). The bacterial pellets are directly taken up in the NuPAGE Novex gel sample buffer (Invitrogen), following the operating mode supplied with the gels, under reduced conditions. The proteins are separated in NuPAGE Novex Bis-Tris 4-12% gel. The Western blot analysis of the monoclonal antibody reactivity for the various domains of annexin A3 is carried out using a chemiluminescent substrate, according to the process described in patent application WO 2009/019365 for example, well known to the person skilled in the art. The antibodies to test were used at a dilution of 10 μg/mL. The exposure time was 100 seconds, unless otherwise specified.

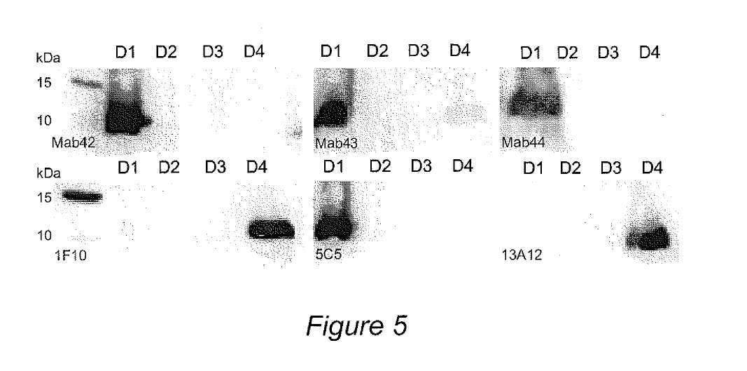

Each anti-ANXA3 antibody was tested with the recombinants expressing the 4 repeat domains of ANXA3; the results of this Western blot analysis are presented in FIG. 5. The monoclonal antibodies TGC42, TGC43, TGC44 and 5C5B10 are specific to domain D1. The monoclonal antibodies 13A12G4H2 and 1F10A6 are directed against domain D4. Antibodies 9C6D4 and 6D9D10, for their part, do not recognise any of the 4 repeat domains, which very probably indicates that their epitopes are situated outside the repeat domains of ANXA3.

Fine Analysis of the Epitopes Recognised by Anti-ANXA3 Antibodies

The determination of the epitopes was performed using the Spotscan technique according to Frank and Döring (20), which is described in detail in patent application WO 2009/019365. To this end, all of the annexin A3 protein sequence was reproduced on a nitrocellulose membrane in the form of peptides of 12 overlapping amino acids, offset by 2 amino acids. Then in a second synthesis, the ANXA3 sequence was reproduced in the form of peptides of 15 overlapping amino acids, offset by one amino acid. The immunoreactivity of these membranes of overlapping peptides was tested with anti-ANXA3 antibodies.

In this way it was possible to delimit more precisely the epitopes of 5 anti-ANXA3 monoclonal antibodies of the 8 studied. The epitopes deduced from the comparison of the overlapping peptide sequences recognised are summarised in Table 6. The minimum epitope is the minimum sequence required to achieve recognition of the antibody, with a more or less intense signal. The optimum epitope is the ideal sequence enabling the best possible recognition of the antibody, including or identical to the minimum epitope. Our results confirm that TGC42 and TGC43 are indeed directed against a single epitope, the one initially described in application WO 2010/034825. Surprisingly, antibody 5C5B10 defines a novel epitope which was not described in the previous state of the art. Antibodies 6D9D 10 and 9C6D4 are specific to the N-terminus of the protein, like monoclonal TGC7 in application WO 2007/141043. Monoclonal antibodies TGC44, 13A12G4H2 and 1F10A6 do not exhibit any reactivity under Spotscan, even on a membrane carrying peptides 20 amino acids long. They probably possess conformational or at least semi-conformational epitopes, whose structures are not sufficiently well reproduced by synthesis peptides.

TABLE 6

| Anti- | | | Minimum |
|---|---|---|---|
| body | Domain | Optimum epitope ^{a} | epitope ^{b} |
| TGC42 | D1 | SNAQRQLIVKEYQAAYG | LIVKEYQAAYG |
| | | (49-65) (SEQ ID: 10) | (55-65) |
| | | | (SEQ ID: 11) |
| TGC43 | D1 | IVKEYQAAYGKE | KEYQAAYG |
| | | (56-67) (SEQ ID: 12) | (58-65) |
| | | | (SEQ ID: 13) |
| 5C5B10 | D1 | DLSGHFEHL | LSGHFEH |
| | | (75-83) (SEQ ID: 14) | (76-82) |
| | | | (SEQ ID: 15) |
| 6D9D10 | N-term | ASIWVGHRGTVRDYPDF | SIWVGHRGTVRD |
| | | SPS | YPDFSP |
| | | (2-21) (SEQ ID: 16) | (3-20) |
| | | | (SEQ ID: 17) |
| 9C6D4 | N-term | YPDF | YPDF |
| | | (15-18) (SEQ ID: 18) | (15-18) |
| | | | SEQ ID: 18) |

^{a} Optimum epitope: Ideal sequence enabling the best possible recognition of the antibody (including or identical to the minimum epitope).
^{b} Minimum epitope: Minimum sequence required to achieve recognition of the antibody (more or less intense signal).

Precisely Locating the Epitope of Antibody TGC44 Using the Novatope Technique

The Novatope system (Merck, Cat No. 69279) is a technology enabling analysis of a protein in order to select domains containing epitopes. The method is based on creating a bank of bacterial clones, each expressing a fragment of the protein, cut at random. These clones are analysed by immunodetection with the antibody that is sought to be characterised. The DNA sequencing of the positive clones makes it possible to deduce the protein sequence of a fragment containing the epitope. The technique was applied following the operating mode supplied with the kit.

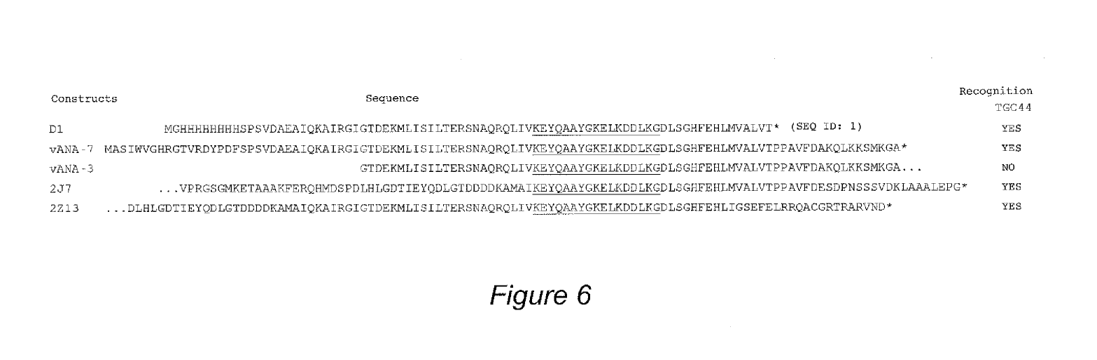

In this way it was possible to isolate and sequence two clones reacting with antibody TGC44. Clone 2J7 expresses sequence KEYQAAYGKELKDDLKGDLSGHFEHLMVALVTPPAVFD (SEQ ID: 19), which corresponds to residues 58-95 of ANXA3. Clone 2Z13 expresses sequence QKAIRGIGTDEKMLISILTERSNAQRQLIVKEYQAAYGKELKDDLKGDLSGHFEHL (SEQ ID: 20), which corresponds to residues 28-83 of ANXA3. The common part between the sequences of these two clones are residues 58-83 of Annexin A3, i.e. sequence KEYQAAYGKELKDDLKGDLSGHFEHL (SEQ ID: 21). Furthermore, since antibodies TGC44 and 5C5B10 are complementary (see example 1), their two epitopes cannot be overlapping, so it is possible to remove the amino acids corresponding to the epitope of antibody 5C5B10, i.e. DLSGHFEHL (75-83) (SEQ ID; 14). So the epitope of antibody TGC44 is within the sequence KEYQAAYGKELKDDLKG (58-74) (SEQ ID: 22). This is the same region as that recognised by TGC42 and TGC43. However, the fact that antibody TGC44 does not exhibit any reactivity under Spotscan indicates a conformational constraint for recognition. TGC44 is able to bind to its epitope only if sequence KEYQAAYGKELKDDLKG (58-74) (SEQ ID: 22) is fused on the N-terminus side to a long sequence of at least 30 residues. In this way the recombinant repeat domain D1, the sequence of which is identified as SEQ ID NO: 6, clones 2J7 and 2Z13 fused to a carrier protein or the recombinant fragment vANA-7 described by application WO 2010/034825, are all recognised by clone TGC44. Conversely, the recombinant fragment vANA-3, which is lacking the first 34 N-terminus amino acids, is not recognised (FIG. 6).

Precisely Locating the Epitope of Antibodies 13A12G4H2 and 1F10A6

The experiment presented in FIG. 5 shows that the epitopes of antibodies 13A12G4H2 and 1F10A6 are within domain D4 of annexin A3. Twelve recombinant proteins were constructed by mutagenesis aimed at improving the “mapping” of the antibodies directed against domain D4. This is the complete sequence of native mature ANXA3 (aa 2-323), fused on the N-terminus side with a histidine tag (non-mutated sequence). PCR mutagenesis was used to introduce Alanine mutations to positions 253, 257, 260, 263, 265, 268, 270, 274, 306, 311 and 317 of the protein sequence (GeneArt Mutagenesis Service, Invitrogen). This technique, known as Alanine scanning, makes it possible to evaluate one by one the importance of the contribution of each mutated amino acid residue of Annexin A3 to the binding of antibodies 13A12G4H2 and 1F10A6. All these DNA fragments were cloned in vector pMRCH79 derived from vector pMR78, and then transformed in bacteria BL21 (DE3). The proteins were produced and purified according to techniques well known to the person skilled in the art, and which were stated at the start of example 3.

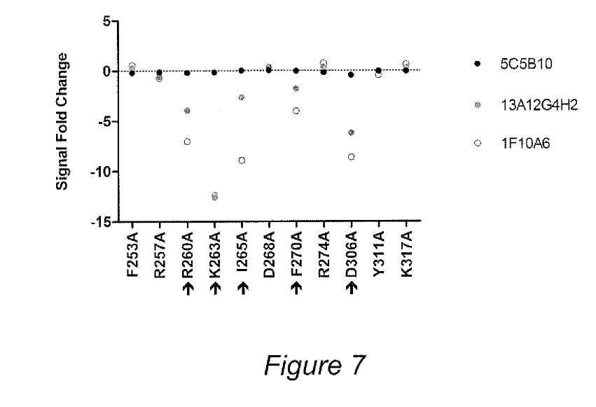

These 12 proteins (11 mutant and 1 non-mutated control) were then used to evaluate the binding capacity of antibodies 13A12G4H2 and 1F10A6 in a sandwich-format ELISA, using antibody TGC44 for capture. Detection antibody 5C5B10, the epitope of which is in domain D1 and which therefore should not be affected by mutations of domain D4, is used as the control. The results are presented in FIG. 7. The graph represents the “signal fold change” (on the Y-axis) of each mutation (on the X-axis). The “signal fold change” corresponds to log₂(mutated protein signal/non-mutated protein signal). The higher the absolute signal fold change value, the more the mutations for which this variation is observed affect the antibody binding. Hence for 5C5B10, the signal fold change is always around 0, indicating that none of the mutations tested disrupt the binding of this monoclonal. By contrast, for 13A12G4H2 and 1F10A6, it was possible to identify amino acids, mutation of which prevents or disrupts binding very significantly. These are positions 260, 263, 265 and 306. Mutation of position 270 has a measurable impact, but less than for the previous positions. This demonstrates that amino acids 260, 263, 265 and 306 of ANXA3 belong to the epitope recognised by monoclonal antibodies 13A12G4H2 and 1F10A6, the two most important residues in the antigen-antibody interaction being Lysine (K) in position 263 and Aspartic Acid (D) in position 306.

### Example 4

### Analytical Specificity of Anti-ANXA3 ELISA Assay Formats

Annexins are a family of proteins that share homologies in terms of function and sequence. A BLAST query conducted on the UniProtKB database, limited to sequences of human origin, was able to identify the proteins with the greatest sequence homology with Annexin A3. In decreasing order of homology, these are annexin A4, annexin A11, annexin A6 and annexin A5.

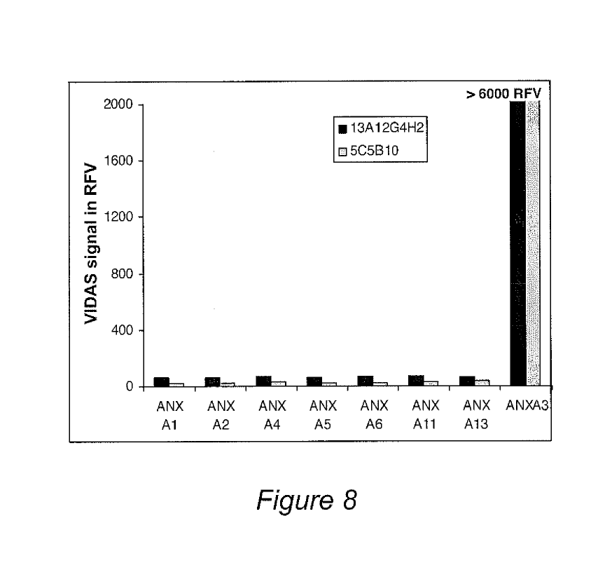

Consequently, it was important to demonstrate the specificity of the ELISA assays to Annexin A3, and the absence of cross reaction with other family members. Since the 3 antibodies TGC42, TGC43 and TGC44 are directed against the same epitope, the TGC44/13A12G4H2 assay (hereafter called simply 13A12G4H2) was chosen as the group 1 prototype assay defined in example 1. Similarly, the TGC44/5C5B10 assay (hereafter called simply 5C5B10) was chosen as the prototype assay representing group 2. The absence of cross reactivity was tested using commercially available antigens, obtained from Abnova: annexin A1 (Cat No. H00000301-P01), annexin A2 (Cat No. H00000302-P01), annexin A4 (Cat No. H00000307-P01), annexin A5 (Cat No. H00000308-P01), annexin A6 (Cat No. H00000309-P01) and annexin All (Cat No. H00000311-P01). Annexin A13 was expressed in recombinant form in the laboratory by cloning in expression vector pMRCH79; fused to a histidine tag, and then purified by metal-chelate affinity chromatography. The proteins were diluted in the buffer of the VIDAS well X1 to a concentration of 12.5 μg/mL for the TGC44/5C5B10 ELISA, and to a concentration of 16 μg/mL for the TGC44/13A12G4H2 ELISA. The results are presented in FIG. 8. The two ELISA formats, TGC44/13A12G4H2 and TGC44/5C5B10, are both highly specific to Annexin A3, and do not exhibit any cross reactivity with the other annexins tested.

### Example 5

### Determining the Affinity of the Anti-Annexin A3 Antibodies

Surface plasmon resonance technology provides a real-time view of the interactions between various unlabelled biomolecules. One of the reagents is bound specifically to a biosensor (sensor chip), while the other species involved in the interaction is in a continuous buffer flow. The surface plasmon resonance measurements were taken using a Biacore T100. The reagents, including the sensor chip CM5, rabbit anti-mouse IgG, specific to the Fc fragment (RAM Fc), and the amine coupling kit for immobilising the antibodies were all obtained from GE-Healthcare Bioscience AB.

In order to study the kinetic characteristics of the binding of the anti-Annexin A3 antibodies, these antibodies were immobilised by capture on the sensor chip, on which the RAM Fc antibody had been covalently coupled in advance. The binding experiments were performed in a buffer, HEPES, at 25° C., with a flow-rate of 30 μL/min. The modification of the resonance signal in RU (Resonance Units) makes it possible to monitor in real time the binding and then the dissociation of the biomolecules on the biosensor surface. Initially, the monoclonal antibody to be studied was injected into channel 2 to obtain a signal of approx. 250 RU. Then Annexin A3 (Arodia) was injected into channels 1 and 2. The association and dissociation times were 5 and 15 minutes respectively. After measuring the resonance responses, the surface of the biosensor was regenerated by washing with 50 mM HCl, at 10 μL/min for 120 seconds. The same measurement process was repeated for each dilution of Annexin A3 protein; in total 9 different dilutions of the protein between 0 and 64 nM were analysed. The sensorgrams obtained were plotted and analysed with the Biacore™ T100 dedicated software, according to the 1:1 interaction model. The kinetic constants of association (Kon) and dissociation (Koff) were measured using the antibodies at a concentration of 3 μg/mL, except for 13A12G4H2 and 1F10A6, which were used at 0.75 μg/mL in order to limit the impact of background noise. The affinity, represented by the dissociation constant (Kd), was calculated (Kd=Koff/Kon).

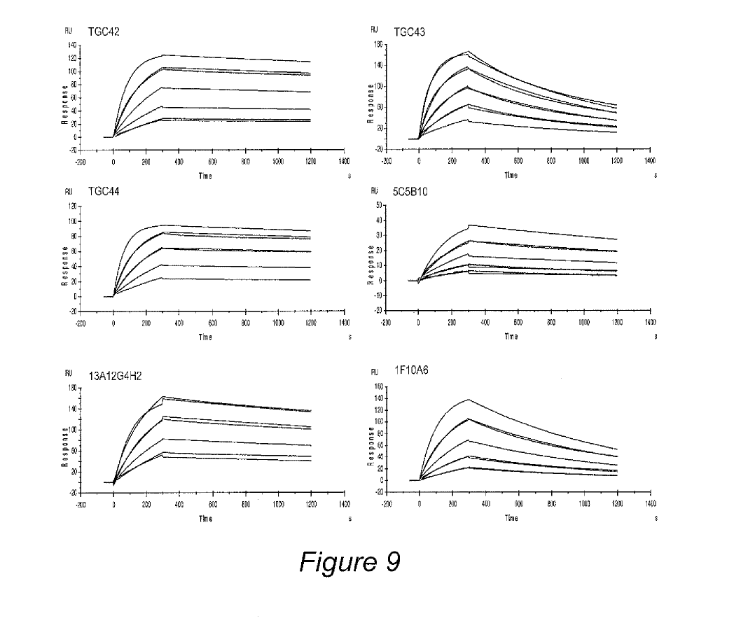

For each anti-Annexin A3 antibody, the graphs representing the resonance signal as a function of time are presented in FIG. 9. The measured association and dissociation constant values, as well as the calculated values of the affinity constants, are shown in Table 7.

All of the anti-ANXA3 antibodies studied exhibit high affinities, with Kd values ranging from 10⁻⁹ to 5×10⁻¹⁰ M. It is possible to classify the antibodies into two distinct groups as a function of their Kd. The first group corresponds to antibodies 13A12G4H2, TGC42 and TGC44, the Kd of which is greater than 10⁻¹⁰ M; this is the antibody group with very high affinity. The second group corresponds to antibodies 5C5B10, 1F10A6 and TGC43, the Kd of which is between 10⁻⁹ and 3.5×10⁻⁹M; this is the antibody group with high affinity.

The three antibodies used for capture, TGC42, TGC43 and TGC44, have equivalent kinetic constants of association, i.e. equivalent capture capacities. Antibody 13A12G4H2 also has a comparable kinetic constant of association, in this way confirming its capture capacity demonstrated by example 1. The kinetic constant of association of antibody 5C5B10 is around 1 log less than that of TGC44, which illustrates its inferior capture capacity.

It is also possible to divide the anti-ANXA3 antibodies into 2 groups in terms of the kinetic constants of dissociation. The first group contains TGC44, TGC42, 13A12G4H2 and 5C5B10; it is characterised by a very low kinetic constant of dissociation, of between 9×10⁻⁵ and 3.5×10⁻⁴ s⁻¹. Once the antigen-antibody binding has occurred, the Annexin A3 is retained by the antibodies of this group, and is not dissociated. The second group contains TGC43 and 1F10A6; it is characterised by a higher kinetic constant of dissociation, of the order of 10⁻³ s⁻¹. The antibodies of this group, even if they manage to bind Annexin A3, dissociate from it much more quickly. In this way, although they have comparable kinetic constants of association, TGC42 and TGC44 are better capture antibodies to use for developing an ELISA assay than TGC43.

TABLE 7

| Antibody bound | | | |
|---|---|---|---|
| on the biosensor | Kon (M⁻¹ · s⁻¹) | Koff (s⁻¹) | Kd (M) |
| 13A12G4H2 | 5.3E+05 | 2.7E−04 | 5.0E−10 |
| TGC42 | 7.3E+05 | 1.0E−04 | 1.8E−10 |
| TGC44 | 9.5E+05 | 9.7E−05 | 1.0E−10 |
| 5C5B10 | 9.8E+04 | 3.4E−04 | 3.4E−09 |
| 1F10A6 | 3.8E+05 | 1.1E−03 | 2.9E−09 |
| TGC43 | 8.4E+05 | 1.1E−03 | 1.3E−09 |

### Example 6

### Use of Prototype ELISA Assays to Distinguish Cancerous and Non-Cancerous in Patient Cohorts

In order to analyse the capacity of Annexin A3 to distinguish patients with a prostate cancer from those free from it, the ELISA assays described in example 1 were used to assay the quantity of Annexin A3 present in the post-digital rectal examination urines of these patients.

The patients included in the “Cancerous” group all have proven prostate cancer, the diagnosis of which was confirmed by histological analysis on a biopsy. For patients included in the “Non-cancerous” or control group, prostate cancer had been ruled out, also by biopsy; the vast majority of these patients have a benign prostatic hyperplasia. The urine samples following digital rectal examination were collected and treated according to the process described in example 1. Two patient cohorts were analysed: Cohort #1 comprises 127 patients, 72 in the “Cancerous” group, and 55 in the “Non-cancerous” group. Cohort #2 comprises 94 patients, 43 in the “Cancerous” group, and 42 in the “Non-cancerous” group. Both cohorts comprise patients with a serous PSA level of between 2.5 and 10 ng/mL. This is the grey area of PSA, in which the clinical performances of the marker are least good.

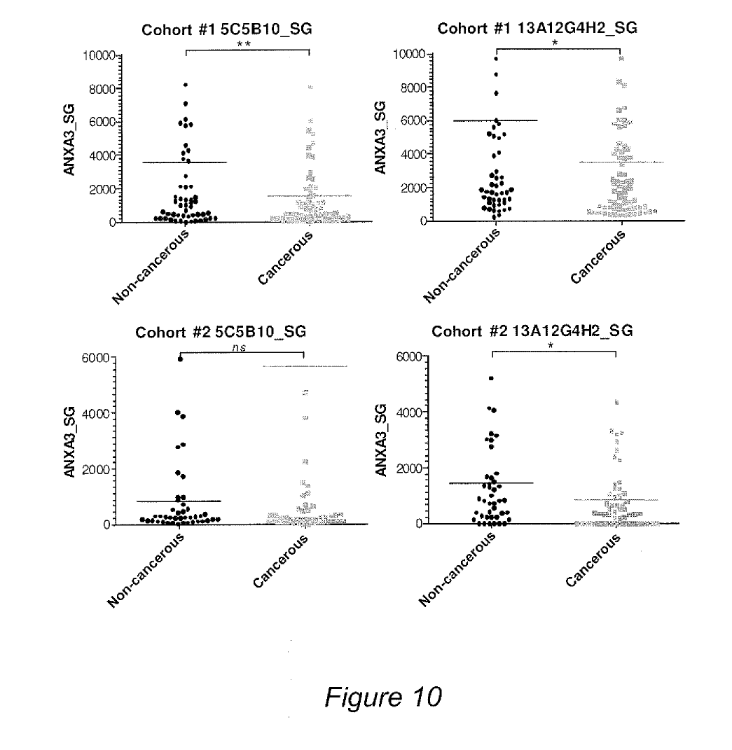

The first ELISA assays performed on urines expressed after digital rectal examination, described in example 1, revealed a previously unsuspected biological complexity. We managed to define two ELISA assay groups which measure a different piece of biological information (partially redundant or not). So it is essential to determine which group of ELISA assays can access the most relevant biological information, i.e. making it possible to best discriminate between “Cancerous” and “Non-cancerous” in a given sampling. The TGC44/13A12G4H2 assay (hereafter called simply 13A12G4H2) was chosen as the prototype assay for group 1 defined in example 1. Similarly, the TGC44/5C5B10 assay (hereafter called simply 5C5B10) was chosen as the prototype assay representing group 2. FIG. 10 shows the values obtained for each of cohorts #1 and #2, with both Annexin A3 ELISA assay formats used. The dose of Annexin A3 was standardised in relation to the urinary density value measured by the Combur 10 strip (Roche Cat No. 04510062171), according to the formula standardised dose=VIDAS dose/(urinary density−1) (21).

In the first patient cohort (cohort #1), both assay formats can distinguish patients with a cancer from the controls. Indeed, the standardised Annexin A3 doses are significantly lower in the “Cancerous” group than in the “Non-cancerous” group, whatever the assay format used (unilateral Mann-Whitney p-value is 0.003 for the TGC44/5C5B10 assay, and 0.02 for the TGC44/13A12G4H2 assay). Conversely, in the second patient cohort (cohort #2), only the assay TGC44/13A12G4H2 format is able to distinguish patients with a cancer from the controls. As with cohort #1, and in accordance with the study performed using the Western blot technique by Schostack et coll. (21), the standardised Annexin A3 doses are significantly lower in the “Cancerous” group than in the “Non-cancerous” group, using the TGC44/13A12G4H2 prototype ELISA (unilateral Mann-Whitney p-value is 0.01). By contrast, the TGC44/5C5B10 prototype is found lacking for this second cohort, and cannot distinguish the patients with a cancer. The TGC44/13A12G4H2 prototype, and more generally the group 1 ELISA formats, therefore seem to be superior in distinguishing prostate cancers. This conclusion matches the ELISPOT analysis described in example 1, which suggests that the group 1 ELISAs are more suitable for detecting ANXA3 of prostatic origin.

### Example 7

### Effect of Calcium Ion and EDTA on the 5C5B10 and 13A12G4H2 Assays

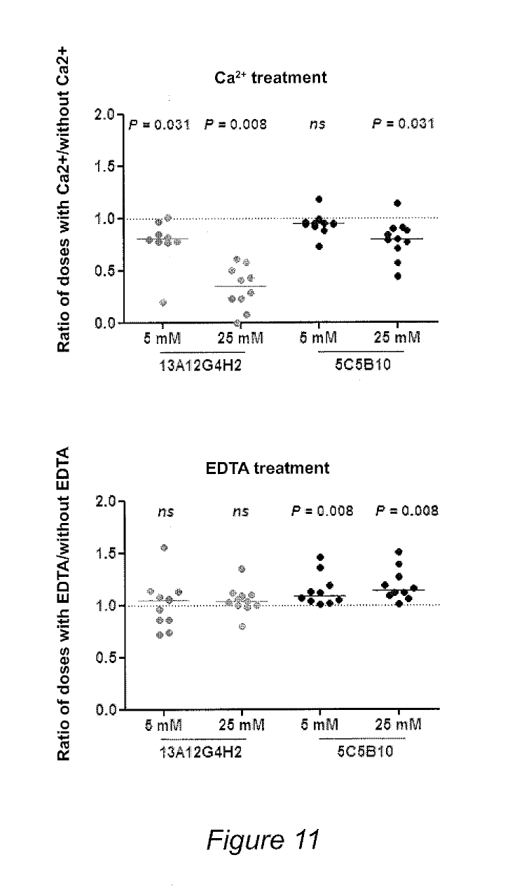

Eleven urines collected after digital rectal examination were assayed directly (without treatment), or after adding 5 or 25 mM of CaCl₂, or after adding 5 or 25 mM of EDTA. The results are presented in FIG. 11. The Y-axis shows the ratio of doses with treatment (Ca₂₊ or EDTA)/dose without treatment, for the 5C5B10 assay and the 13A12G4H2 assay. The addition of the calcium ion to the urines progressively lowers the doses measured by the 13A12G4H2 assay, the more the added calcium concentration is increased, the lower the measured dose. The 5C5B10 assay is much less affected by the presence of calcium in the urines, even if at high concentration of calcium a slight effect is observed. As for EDTA treatment of urines, it has no effect on the 13A12G4H2 assay, but for the 5C5B10 assay, it causes a very slight increase in the measured doses. The introduction of EDTA or a similar chelating agent to urines before assaying, via buffers or in solid form, formulated or non-formulated, therefore makes it possible to improve the assays, in particular the 13A12G4H2 assay, by reducing the calcium ion effect on the ANXA3 doses.

### BIBLIOGRAPHIC REFERENCES

1. Ferlay, J., Parkin, D. M., and Steliarova-Foucher, E., Estimates of cancer incidence and mortality in Europe in 2008, Eur J Cancer, 46, 765.
2. Barton, G. J., Newman, R. H., Freemont, P. S., and Crumpton, M. J., Amino acid sequence analysis of the annexin super-gene family of proteins, Eur J Biochem, 198, 749 (1991).
3. Le Cabec, V., Russo-Marie, F., and Maridonneau-Parini, I., Differential expression of two forms of annexin 3 in human neutrophils and monocytes and along their differentiation, Biochem Biophys Res Commun, 189, 1471 (1992).
4. Le Cabec, V., and Maridonneau-Parini, I., Annexin 3 is associated with cytoplasmic granules in neutrophils and monocytes and translocates to the plasma membrane in activated cells, Biochem J, 303 (Pt 2), 481 (1994).
5. Bianchi, C., Bombelli, S., Raimondo, F., Torsello, B., Angeloni, V., Ferrero, S., Di Stefano, V., Chinello, C., Cifola, I., Invernizzi, L., Brambilla, P., Magni, F., Pitto, M., Zanetti, G., Mocarelli, P., and Perego, R. A., Primary cell cultures from human renal cortex and renal-cell carcinoma evidence a differential expression of two spliced isoforms of Annexin A3, Am J Pathol, 176, 1660.
6. Kollermann, J., Schlomm, T., Bang, H., Schwall, G. P., von Eichel-Streiber, C., Simon, R., Schostak, M., Huland, H., Berg, W., Sauter, G., Klocker, H., and Schrattenholz, A., Expression and prognostic relevance of annexin A3 in prostate cancer, Eur Urol, 54, 1314 (2008).
7. Wozny, W., Schroer, K., Schwall, G. P., Poznanovic, S., Stegmann, W., Dietz, K., Rogatsch, H., Schaefer, G., Huebl, H., Klocker, H., Schrattenholz, A., and Cahill, M. A., Differential radioactive quantification of protein abundance ratios between benign and malignant prostate tissues: cancer association of annexin A3, Proteomics, 7, 313 (2007).
8. Kohler, G., and Milstein, C., Continuous cultures of fused cells secreting antibody of predefined specificity, Nature, 256, 495 (1975).
9. Kohler, G., Howe, S. C., and Milstein, C., Fusion between immunoglobulin-secreting and nonsecreting myeloma cell lines, Eur J Immunol, 6, 292 (1976).
10. Webber, M. M., Quader, S. T., Kleinman, H. K., Bello-DeOcampo, D., Storto, P. D., Bice, G., DeMendonca-Calaca, W., and Williams, D. E., Human cell lines as an in vitro/in vivo model for prostate carcinogenesis and progression, Prostate, 47, 1 (2001).
11. Rehman, I., Azzouzi, A. R., Catto, J. W., Allen, S., Cross, S. S., Feeley, K., Meuth, M., and Hamdy, F. C., Proteomic analysis of voided urine after prostatic massage from patients with prostate cancer: a pilot study, Urology, 64, 1238 (2004).
12. Clark, H. F., Geldreich, E. E., Jeter, H. L., and Kabler, P. W., The membrane filter in sanitary bacteriology, Public Health Rep, 66, 951 (1951).
13. Kabler, P. W., and Clark, H. F., The use of differential media with the membrane filter, Am J Public Health Nations Health, 42, 390 (1952).
14. Cheruvanky, A., Zhou, H., Pisitkun, T., Kopp, J. B., Knepper, M. A., Yuen, P. S., and Star, R. A., Rapid isolation of urinary exosomal biomarkers using a nanomembrane ultrafiltration concentrator, Am J Physiol Renal Physiol, 292, F1657 (2007).
15. Pan, B. T., Teng, K., Wu, C., Adam, M., and Johnstone, R. M., Electron microscopic evidence for externalization of the transferrin receptor in vesicular form in sheep reticulocytes, J Cell Biol, 101, 942 (1985).
16. Wolfers, J., Lozier, A., Raposo, G., Regnault, A., Thery, C., Masurier, C., Flament, C., Pouzieux, S., Faure, F., Tursz, T., Angevin, E., Amigorena, S., and Zitvogel, L., Tumor-derived exosomes are a source of shared tumor rejection antigens for CTL cross-priming, Nat Med, 7, 297 (2001).
17. Raposo, G., Nijman, H. W., Stoorvogel, W., Liejendekker, R., Harding, C. V., Melief, C. J., and Geuze, H. J., B lymphocytes secrete antigen-presenting vesicles, J Exp Med, 183, 1161 (1996).
18. Thery, C., Amigorena, S., Raposo, G., and Clayton, A., Isolation and characterization of exosomes from cell culture supernatants and biological fluids, Curr Protoc Cell Biol, Chapter 3, Unit 3 22 (2006).
19. Rubartelli, A., Cozzolino, F., Talio, M., and Sitia, R., A novel secretory pathway for interleukin-1 beta, a protein lacking a signal sequence, EMBO J, 9, 1503 (1990).
20. Frank, R., and Döring, R., Simultaneous multiple peptide synthesis under continuous flow conditions on cellulose papers discs as segmental solid synthesis, Tetrahedron, 44, 6031 (1988).
21. Schostak, M., Schwall, G. P., Poznanovic, S., Groebe, K., Muller, M., Messinger, D., Miller, K., Krause, H., Pelzer, A., Horninger, W., Klocker, H., Hennenlotter, J., Feyerabend, S., Stenzl, A., and Schrattenholz, A., Annexin A3 in urine: a highly specific noninvasive marker for prostate cancer early detection, J Urol, 181, 343 (2009).

1. A process for in vitro diagnosis of a prostate cancer, according to which a urine sample to be analysed is contacted with two antibodies, a capture antibody and a detection antibody, one of the two antibodies is directed against the first repeat domain of native human Annexin A3, the sequence of which is identified as SEQ ID NO: 1, and the other of the two antibodies is directed against the fourth repeat domain of native human Annexin A3, the sequence of which is identified as SEQ ID NO: 2.

2. The process according to claim 1, in which the antibody directed against the first repeat domain of native human Annexin A3 is chosen from the antibodies directed against an epitope, the amino acid sequence of which comprises at least 7 consecutive amino acids, and no more than 17 consecutive amino acids of SEQ ID NO: 1.

3. The process according to claim 1, in which the antibody directed against the first repeat domain of native human Annexin A3 is chosen from the antibodies directed against a polypeptide included in SEQ ID NO: 1, the amino acid sequence of which is selected from the following sequences:
SNAQRQLIVKEYQAAYG (SEQ ID NO: 10),
LIVKEYQAAYG (SEQ ID NO: 11)
IVKEYQAAYGKE (SEQ ID NO: 12),
KEYQAAYG (SEQ ID NO: 13),
DLSGHFEHL (SEQ ID NO: 14),
LSGHFEH (SEQ ID NO: 15),
and
KEYQAAYGKELKDDLKG (SEQ ID NO: 22), provided that the amino acid sequence SEQ ID NO: 22 is fused on the N-terminus side to a sequence of at least 30 amino acids.

4. The process according to claim 1, in which the antibody directed against the fourth repeat domain of native human Annexin A3 is chosen from the antibodies directed against an epitope, the amino acid sequence of which comprises at least 7 consecutive amino acids, and no more than 50 consecutive amino acids of SEQ ID NO: 2.

5. The process according to claim 4, in which the antibody directed against the fourth repeat domain of native human Annexin A3 is chosen from the antibodies directed against an epitope, the amino acid sequence of which comprises at least 7 consecutive amino acids, and no more than 45 consecutive amino acids of SEQ ID NO: 2.

6. The process according to claim 4, in which the antibody directed against the fourth repeat domain of native human Annexin A3 is chosen from the antibodies directed against an epitope which is included in an amino acid sequence corresponding to the amino acid sequence starting at residue 3 and ending at residue 49 of SEQ ID NO: 2.

7. The process according to claim 4, in which the epitope comprises in position 6 of SEQ ID NO: 2 a Lys residue.

8. The process according to claim 4, in which the epitope comprises in position 6 of SEQ ID NO: 2 a Lys residue and in position 49 of SEQ ID NO: 2 an Asp residue.

9. The process according to claim 8, in which the epitope comprises in position 7 of SEQ ID NO: 2 a Gly residue, in position 8 of SEQ ID NO: 2 an Ile residue, and in position 9 of SEQ ID NO: 2 a Gly residue.

10. The process according to claim 4, in which the epitope comprises in position 3 of SEQ ID NO: 2 an Arg residue, in position 6 of SEQ ID NO: 2 a Lys residue, in position 7 of SEQ ID NO: 2 a Gly residue, in position 8 of SEQ ID NO: 2 an Ile residue, in position 9 of SEQ ID NO: 2 a Gly residue, and in position 49 of SEQ ID NO: 2 an Asp residue.

11. The process according to claim 1, in which the antibody directed against the first repeat domain of native human Annexin A3, the sequence of which is identified as SEQ ID NO: 1, is the capture antibody and the antibody directed against the fourth repeat domain of native human Annexin A3, the sequence of which is identified as SEQ ID NO: 2, is the detection antibody.

12. The process according to claim 1, in which the capture antibody and detection antibody are antibodies which exhibit a high affinity, with an affinity constant of at least 10⁻⁹.

13. The process according to claim 1, in which the capture antibody and detection antibody are antibodies which exhibit a low dissociation constant of less than 2×10⁻³ s⁻¹.

14. An immunoassay kit for in vitro diagnosis of a prostate cancer in a urine sample to be analysed, comprising two antibodies, a capture antibody and a detection antibody, one of the two antibodies being directed against the first repeat domain of native human Annexin A3, the sequence of which is identified as SEQ ID NO: 1, and the other of the two antibodies being directed against the fourth repeat domain of native human Annexin A3, the sequence of which is identified as SEQ ID NO: 2.

15. A kit according to claim 14, in which the antibody directed against the first repeat domain of native human Annexin A3 is chosen from the antibodies directed against an epitope, the amino acid sequence of which comprises at least 7 consecutive amino acids, and no more than 17 consecutive amino acids of SEQ ID NO: 1.

16. A kit according to claim 14, in which the antibody directed against the first repeat domain of native human Annexin A3 is chosen from the antibodies directed against a polypeptide included in SEQ ID NO: 1, the amino acid sequence of which is selected from the following sequences:
SNAQRQLIVKEYQAAYG (SEQ ID NO: 10),
LIVKEYQAAYG (SEQ ID NO: 11)
IVKEYQAAYGKE (SEQ ID NO: 12),
KEYQAAYG (SEQ ID NO: 13),
DLSGHFEHL (SEQ ID NO: 14),
LSGHFEH (SEQ ID NO: 15),
and
KEYQAAYGKELKDDLKG (SEQ ID NO: 22), provided that the amino acid sequence SEQ ID NO: 22 is fused on the N-terminus side to a sequence of at least 30 amino acids.

17. A kit according to claim 14, in which the antibody directed against the fourth repeat domain of native human Annexin A3 is chosen from the antibodies directed against an epitope, the amino acid sequence of which comprises at least 7 consecutive amino acids, and no more than 50 consecutive amino acids of SEQ ID NO: 2.

18. A kit according to claim 17, in which the antibody directed against the fourth repeat domain of native human Annexin A3 is chosen from the antibodies directed against an epitope, the amino acid sequence of which comprises at least 7 consecutive amino acids, and no more than 45 consecutive amino acids of SEQ ID NO: 2.

19. A kit according to claim 17, in which the antibody directed against the fourth repeat domain of native human Annexin A3 is chosen from the antibodies directed against an epitope which is included in an amino acid sequence corresponding to the amino acid sequence starting at residue 3 and ending at residue 49 of SEQ ID NO: 2.

20. A kit according to claim 17, in which the antibody directed against the fourth repeat domain of native human Annexin A3 is chosen from the antibodies directed against an epitope, said epitope comprising in position 6 of SEQ ID NO: 2 a Lys residue.

21. A kit according to claim 17, in which the antibody directed against the fourth repeat domain of native human Annexin A3 is chosen from the antibodies directed against an epitope, said epitope comprising in position 6 of SEQ ID NO: 2 a Lys residue and in position 49 of SEQ ID NO: 2 an Asp residue.

22. A kit according to claim 21, in which the antibody directed against the fourth repeat domain of native human Annexin A3 is chosen from the antibodies directed against an epitope, said epitope comprising in position 7 of SEQ ID NO: 2 a Gly residue, in position 8 of SEQ ID NO: 2 an Ile residue and in position 9 of SEQ ID NO: 2 a Gly residue.

23. A kit according to claim 17, in which the antibody directed against the fourth repeat domain of native human Annexin A3 is chosen from the antibodies directed against an epitope, said epitope comprising in position 3 of SEQ ID NO: 2 an Arg residue, in position 6 of SEQ ID NO: 2 a Lys residue, in position 7 of SEQ ID NO: 2 a Gly residue, in position 8 of SEQ ID NO: 2 an Ile residue, in position 9 of SEQ ID NO: 2 a Gly residue, and in position 49 of SEQ ID NO: 2 an Asp residue.

24. A kit according to claim 14, comprising the capture antibody, which is directed against the first repeat domain of native human Annexin A3, the sequence of which is identified as SEQ ID NO: 1, and the detection antibody, which is directed against the fourth repeat domain of native human Annexin A3, the sequence of which is identified as SEQ ID NO: 2.

25. A kit according to claim 14, in which the capture antibody and the detection antibody are antibodies which exhibit a high affinity, with an affinity constant of at least 10⁻⁹.

26. A kit according to claim 14, in which the capture antibody and detection antibody are antibodies which exhibit a low dissociation constant of less than 2×10⁻³ s⁻¹.
